(19) United States
(12) Patent Application Publication
(10) Pub. No.: US 2025/0360065 A1
(43) Pub. Date: Nov. 27, 2025

(54) LOW-VISCOSITY MULTI-TYPE EMULSION COMPOSITION

(71) Applicant: AMOREPACIFIC CORPORATION, Seoul (KR)

(72) Inventors: Yoonkyun HWANG, Yongin-si (KR); Myoungwoo KIM, Yongin-si (KR); Eunmi KIM, Yongin-si (KR)

(73) Assignee: AMOREPACIFIC CORPORATION, Seoul (KR)

(21) Appl. No.: 19/206,286

(22) Filed: May 13, 2025

(30) Foreign Application Priority Data
May 22, 2024 (KR) 10-2024-0066614

(51) Int. Cl. A61K 8/06; A61Q 19/00

(52) U.S. Cl. CPC A61K 8/066; A61Q 19/00

(57) ABSTRACT

A multi-type emulsion composition and a preparation method thereof are disclosed. The emulsion composition is a composition in which a water part and an oil part are mixed and emulsified, the water part includes a non-amphipathic nanoparticle dispersion, the oil part includes an emulsion and the emulsion includes an oil phase or silicone phase as an external phase, the emulsion composition includes the water part as a continuous phase and the oil part as a dispersed phase, and the dispersed phase is surrounded by the non-amphipathic nanoparticles.

### CROSS REFERENCE TO RELATED APPLICATION

The present application claims priority to Korean Patent Application No. 10-2024-0066614, filed May 22, 2024, the entire contents of which are hereby incorporated by this reference.

### BACKGROUND OF THE INVENTION

### Field of the Invention

The present disclosure discloses a multi-type emulsion composition and a preparation method thereof.

### Description of the Related Art

In the preparation of a w/o/w multi-type emulsion formulation, a w/o emulsion is generally prepared in the first step, an o/w emulsion is prepared in the second step, and then a w/o/w formulation is finally produced. The multi-type emulsion formulation is itself in a thermodynamically unstable state because the internal pressure where the w/o is dispersed is higher than the pressure in the continuous phase (external phase) to create a force that causes the internal liquid to move to the outside, making the formulation unstable. To overcome or delay this phenomenon, research has been conducted to prevent internal particles from escaping by adjusting osmotic pressure, forming a lamellar structure, or increasing hardness, but the stability of the multi-type emulsion formulation is still insufficient. To address this unstable formulation stability problem, PEG surfactants with high HLB values which mean excellent emulsion capacity are generally used or the viscosity and/or hardness of the contents are/is increased in the related art. However, since PEG may produce harmful components such as ethylene oxide and 1,4-dioxane during the preparation process, which may cause skin problems or hives, research using other surfactants has been actively conducted as an alternative.

### SUMMARY OF THE INVENTION

In one aspect, an object of the present disclosure is to provide a multi-type emulsion composition.

In another aspect, an object of the present disclosure is to provide a method for preparing the emulsion composition.

In one aspect, the present disclosure provides a multi-type emulsion composition, wherein the emulsion composition is formed by mixing and emulsifying a water part and an oil part, the water part includes a non-amphipathic nanoparticle dispersion, the oil part includes an emulsion, the emulsion includes an oil phase or silicone phase as an external phase, the emulsion composition includes the water part as a continuous phase and the oil part as a dispersed phase, and the dispersed phase is surrounded by the non-amphipathic nanoparticles.

In an exemplary embodiment, the non-amphipathic nanoparticles may have an average size of 10 nm or more and less than 1 μm.

In an exemplary embodiment, the non-amphipathic nanoparticle may be selected from the group consisting of a nanoemulsion particle, a solid lipid nanoparticle (SLN), a liposome, and a polymersome.

In an exemplary embodiment, the non-amphipathic nanoparticle dispersion may be immiscible with the oil part and/or immiscible with the oil phase or silicone phase.

In an exemplary embodiment, the emulsion may include a water phase as an internal phase.

In an exemplary embodiment, the non-amphipathic nanoparticle dispersion and the emulsion may be included at a weight ratio of more than 1:1.

In an exemplary embodiment, the non-amphipathic nanoparticle dispersion may be included in an amount of less than 25 wt % based on a total weight of the emulsion composition.

In an exemplary embodiment, the emulsion may be included in an amount of less than 15 wt % based on a total weight of the emulsion composition.

In an exemplary embodiment, the dispersed phase may have an average size of 1 to 50 μm.

In an exemplary embodiment, the emulsion composition may further include a surfactant.

In an exemplary embodiment, the emulsion composition may be PEG-free.

In an exemplary embodiment, the emulsion composition may have a viscosity of 4,000 to 14,000 cps.

In an exemplary embodiment, the emulsion composition may be formed by mixing and emulsifying the water part and the oil part at room temperature.

In an exemplary embodiment, the emulsion composition may be a w/o/w or w/s/w formulation.

In another aspect, the present disclosure provides a method for preparing the multi-type emulsion composition, the method including: preparing a non-amphipathic nanoparticle dispersion; preparing an emulsion including an oil phase or silicone phase as an external phase; preparing a water part including the non-amphipathic nanoparticle dispersion; preparing an oil part including the emulsion; and mixing the water part and the oil part.

In one aspect, a technology disclosed in the present disclosure has an effect of providing a multi-type emulsion composition.

In another aspect, a technology disclosed in the present disclosure has an effect of providing a method for preparing the emulsion composition.

The multi-type emulsion technology in the related art includes a method of improving stability by adjusting the selection and proportion of surfactants, a method of reducing the fluidity of emulsion particles by additionally using an oil gelling agent, and/or a method of controlling the fluidity using a thickener to prevent coalescence. Although the corresponding method can prepare a multi-type emulsion formulation, there is a disadvantage in that a unique residual feeling is produced according to the use of surfactants, oil gelling agents and/or thickeners. Further, there is a disadvantage in that the content of the surfactant needs to be increased in order to improve stability. The present disclosure has the effect of improving the feeling of use of a multi-type emulsion formulation and forming a multi-type emulsion formulation, particularly a low-viscosity multi-type emulsion formulation, without using a surfactant or while minimizing the use of the surfactant.

### BRIEF DESCRIPTION OF THE DRAWINGS

FIG. 1 illustrates a photograph of the emulsion composition of Example 1-4 taken with an optical microscope (DSX110, OLYMPUS) (scale bar 20 μm).

FIG. 2 illustrates a photograph of the emulsion composition of Example 1-4 taken with an optical microscope (DSX110, OLYMPUS) (scale bar 5 μm).

FIG. 3 illustrates a photograph of the emulsion compositions of Example 1-4, which confirms the formulation stability of the emulsion compositions and is taken at each storage temperature.

FIG. 4 illustrates a photograph of the emulsion compositions of Comparative Example 1-6, which confirms the formulation stability of the emulsion compositions and is taken at each storage temperature.

FIG. 5 illustrates a photograph of the emulsion compositions of Comparative Examples 1-6 taken from different angles.

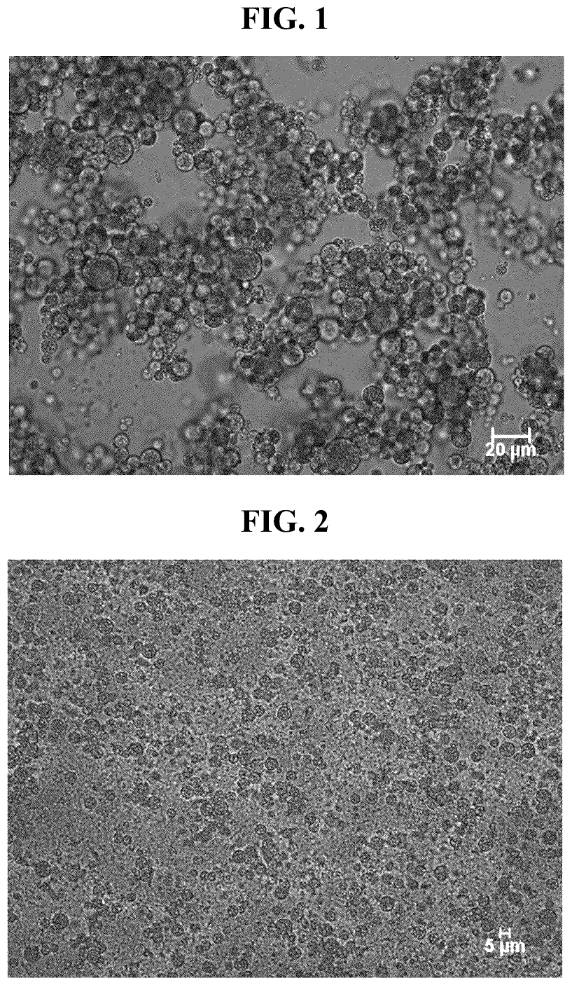

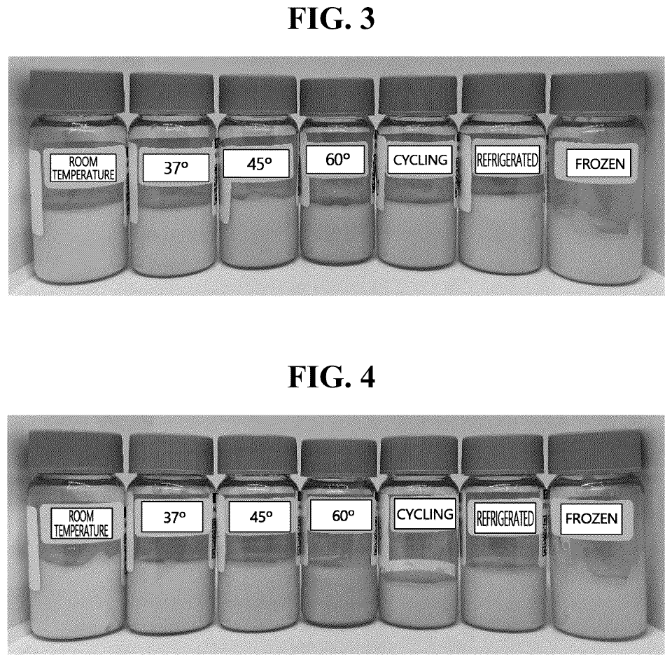

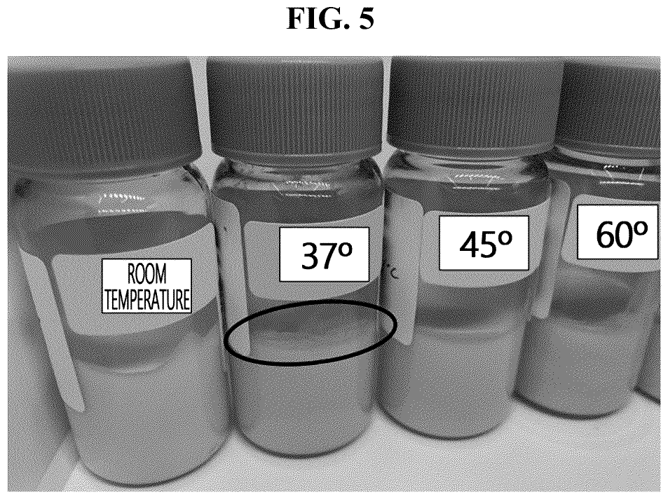

### DETAILED DESCRIPTION OF THE INVENTION

Hereinafter, the present disclosure will be described in detail.

In one aspect, the present disclosure provides a multi-type emulsion composition, wherein the emulsion composition is a composition in which a water part and an oil part are mixed and emulsified, the water part includes a non-amphipathic nanoparticle dispersion, the oil part includes an emulsion and the emulsion includes an oil phase or silicone phase as an external phase, the emulsion composition includes the water part as a continuous phase and the oil part as a dispersed phase, and the dispersed phase is surrounded by the non-amphipathic nanoparticles.

In an exemplary embodiment, the water part and the oil part may be mixed at a weight ratio of 70 to 90:10 to 30, 72 to 88:12 to 28, 76 to 88:12 to 24, or 78 to 86:14 to 22.

The continuous phase refers to a phase that is continuous, and the dispersed phase refers to a phase that is dispersed in the continuous phase.

The non-amphipathic nanoparticles refer to nanoparticles that are not amphipathic. Amphiphilic means having both hydrophilic and hydrophobic parts. The physical properties of the nanoparticles are not amphipathic. For example, the non-amphipathic nanoparticles according to the present disclosure may be formed using an amphipathic substance such as a phospholipid, but the physical properties of the formed nanoparticles themselves do not have amphipathic properties. Therefore, there is no need for modifying the physical properties so as to have a Janus structure, and there is a difference from Pickering emulsion compositions in the related art, in which a powder with a Janus structure is used.

In an exemplary embodiment, the non-amphipathic nanoparticle dispersion may be an aqueous dispersion in which non-amphipathic nanoparticles are dispersed.

In an exemplary embodiment, the non-amphipathic nanoparticle dispersion may include non-amphipathic nanoparticles in the dispersion at a concentration (w/w) of 15% or less, or at a concentration (w/w) of 5% to 15%. Within the above range, the concentration may be adjusted according to the content of the oil part. In another exemplary embodiment, the non-amphipathic nanoparticle dispersion may include non-amphipathic nanoparticles in the dispersion at a concentration (w/w) of 1% or more, 2% or more, 3% or more, 4% or more, 5% or more, 6% or more, 7% or more, 8% or more, 9% or more, 10% or more, 11% or more, 12% or more, 13% or more, or 14% or more, and 15% or less, 14% or less, 13% or less, 12% or less, 11% or less, 10% or less, 9% or less, 8% or less, 7% or less, or 6% or less in order to implement a low-viscosity multi-type emulsion formulation and improve the emulsion stability of the multi-type emulsion formulation.

In an exemplary embodiment, the non-amphipathic nanoparticles may have a nanoscale size.

In an exemplary embodiment, the non-amphipathic nanoparticles may have an average size of 10 nm or more and less than 1 μm.

In another exemplary embodiment, the non-amphipathic nanoparticles may have an average size of 10 nm or more, 50 nm or more or 100 nm or more, and less than 1 μm, 900 nm or less, 800 nm or less, 700 nm or less, 600 nm or less, 500 nm or less, 400 nm or less, 300 nm or less, or 200 nm or less. For example, the non-amphipathic nanoparticles may have an average size of 10 nm to 700 nm or 10 nm to 500 nm.

In still another exemplary embodiment, the non-amphipathic nanoparticles may have an average size of 500 nm or less, or 150 to 300 nm.

In an exemplary embodiment, the size may refer to the diameter of the particle.

In an exemplary embodiment, the diameter may refer to the longest diameter.

In an exemplary embodiment, the non-amphipathic nanoparticle may be selected from the group consisting of a nanoemulsion particle, a solid lipid nanoparticle (SLN), a liposome, and a polymersome. The nanoemulsion particle, solid lipid nanoparticle, liposome, and polymersome may be prepared and used by typical preparation methods known in the art.

Generally, emulsions may be divided into microemulsions, nanoemulsions, and macroemulsions according to the average size of the internal phase. The nanoemulsion refers to an emulsion of two or more immiscible liquids, one of which is dispersed in the other in a state of small particles, with the size of the particles being in a nanometer unit size. The nanoemulsion particles refer to the internal phase of the nanoemulsion, that is, emulsion particles.

The solid lipid nanoparticles are known as one of the drug delivery systems proposed to overcome the disadvantages of colloidal carriers in the related art, and the size thereof may be determined by various factors such as the type and amount of lipid used and the type and amount of surfactant used.

The liposome is a spherical or ellipsoidal structure formed from lipids, and is characterized by having an internal space separated from the outside by one or more bilayer membranes. For example, the liposome may have a structure of a bilayer membrane which is spontaneously arranged by interactions between molecules that have both lipophilicity and hydrophilicity, such as phospholipids.

The polymersomes are similar in structure to liposomes, and have a membrane surrounding an internal fluid, and the membrane may include a polymer. For example, a structure of a molecular bilayer membrane may be formed by the self-association of amphipathic copolymers similar to phospholipids.

In an exemplary embodiment, the non-amphipathic nanoparticles may include an effective ingredient (also referred to as an active ingredient).

The effective ingredient may include effective ingredients that can be dissolved in oil, such as vitamin A, for example, retinol, vitamin E, carotene, coenzyme Q10, resveratrol, beta-carotene, bakuchiol, and lycopene.

In an exemplary embodiment, the non-amphipathic nanoparticles may no differentiation between an inner layer and an outer layer or may have a layered structure with two or more layers.

In an exemplary embodiment, the effective ingredient may be carried in non-amphipathic nanoparticles. That is, when the non-amphipathic nanoparticles have a layered structure of two or more layers, the inner layer may carry an effective ingredient such as whitening, wrinkle amelioration, and antioxidant properties. In an exemplary embodiment, the non-amphipathic nanoparticles may carry a water-soluble or oil-soluble effective ingredient.

In an exemplary embodiment, the non-amphipathic nanoparticles may be amorphous, or may have a shape such as spherical or ellipsoidal.

In an exemplary embodiment, the non-amphipathic nanoparticle dispersion may be immiscible with the oil part and/or immiscible with the oil phase or silicone phase.

In an exemplary embodiment, the emulsion may be a liquid emulsion.

In an exemplary embodiment, the emulsion may include a water phase as an internal phase.

In an exemplary embodiment, the emulsion may be a w/o or w/s formulation. The multi-type emulsion composition of the present disclosure according to one aspect has an effect of significantly improving the emulsion stability of the multi-type emulsion formulation by first preparing a w/o or w/s emulsion, then adding the w/o or w/s emulsion to an oil part, and then mixing and emulsifying the oil part with a water part to which a non-amphipathic nanoparticle dispersion is added.

In an exemplary embodiment, the emulsion may be a w/o formulation, and the emulsion composition may be a w/o/w formulation.

In an exemplary embodiment, the emulsion may be a w/s formulation, and the emulsion composition may be a w/s/w formulation. In this case, the oil part may be a silicone part. In the present application, the oil part may refer to a silicone part. For example, the oil part may include an elastomer, an silicone oil, and the like. In an exemplary embodiment, the elastomer may include one or more selected from the group consisting of vinyl dimethicone/methicone silsesquioxane crosspolymer, POLYSILICONE-11, POLYSILICONE-13, diphenyl dimethicone/vinyl diphenyl dimethicone/silsesquioxane crosspolymer, dimethicone/vinyl dimethicone crosspolymer, dimethicone/phenyl vinyl dimethicone crosspolymer, and polymethylsilsesquioxane. In an exemplary embodiment, the silicone oil may include one or more selected from the group consisting of a methicone-based silicone oil, a dimethicone-based silicone oil, a cyclomethicone-based silicone oil, and a phenyl trimethicone-based silicone oil.

In an exemplary embodiment, the non-amphipathic nanoparticle dispersion and the emulsion may be included at a weight ratio of more than 1:1, a weight ratio of more than 1 to 8:1, a weight ratio of more than 1 to 7.5:1, a weight ratio of more than 1 to 7:1, a weight ratio of more than 1 to 6.5:1, a weight ratio of more than 1 to 6:1, a weight ratio of more than 1 to 5.5:1, a weight ratio of more than 1 to 5:1, a weight ratio of more than 1 to 4.5:1, a weight ratio of more than 1 to 4:1, a weight ratio of more than 1 to 3.5:1, or a weight ratio of more than 1 to 3:1.

In another exemplary embodiment, the non-amphipathic nanoparticle dispersion and the emulsion may be included at a weight ratio of 1.5 or more: 1, a weight ratio of 1.5 to 8:1, a weight ratio of 1.5 to 7.5:1, a weight ratio of 1.5 to 7:1, a weight ratio of 1.5 to 6.5:1, a weight ratio of 1.5 to 6:1, a weight ratio of 1.5 to 5.5:1, a weight ratio of 1.5 to 5:1, a weight ratio of 1.5 to 4.5:1, a weight ratio of 1.5 to 4:1, a weight ratio of 1.5 to 3.5:1, or a weight ratio of 1.5 to 3:1.

In an exemplary embodiment, the non-amphipathic nanoparticle dispersion may be included in an amount of less than 25 wt %, 24 wt % or less, 23 wt % or less, 22 wt % or less, 21 wt % or less, 20 wt % or less, 19 wt % or less, 18 wt % or less, 17 wt % or less, 16 wt % or less, or 15 wt % or less based on the total weight of the emulsion composition. In another exemplary embodiment, the non-amphipathic nanoparticle dispersion may be included in an amount of 5 wt % or more, 6 wt % or more, 7 wt % or more, 8 wt % or more, 9 wt % or more, or 10 wt % or more based on the total weight of the emulsion composition. When the content of the non-amphipathic nanoparticle dispersion is low, the emulsion stability of the multi-type emulsion formulation may deteriorate, and when the content of the non-amphipathic nanoparticle dispersion is high, the emulsion stability may be enhanced, but it may be difficult to form a low-viscosity multi-type emulsion formulation. Therefore, the non-amphipathic nanoparticle dispersion may be preferably included in an amount of 5 wt % or more and less than 25 wt %, 5 to 24 wt %, 5 to 23 wt %, 5 to 22 wt %, 5 to 21 wt %, or 5 to 20 wt % based on the total weight of the emulsion composition.

In an exemplary embodiment, the emulsion may be included in an amount of less than 15 wt %, 14 wt % or less, 13 wt % or less, 12 wt % or less, 11 wt % or less, 10 wt % or less, 9 wt % or less, 8 wt % or less, 7 wt % or less, 6 wt % or less, 5 wt % or less, or 4 wt % or less based on the total weight of the emulsion composition. In another exemplary embodiment, the emulsion may be included in an amount of more than 3 wt %, 4 wt % or more, 5 wt % or more, 6 wt % or more, 7 wt % or more, 8 wt % or more, 9 wt % or more, or 10 wt % or more based on the total weight of the emulsion composition. When the content of the emulsion is low, it may be difficult to provide the unique feeling of use of the multi-type emulsion formulation, and when the content of the emulsion is high, the emulsion stability of the multi-type emulsion formulation may deteriorate, and a heavy and thick feeling of use may occur. Therefore, the emulsion may be preferably included in an amount of more than 3 wt % and less than 15 wt %, 4 to 14 wt %, 5 to 13 wt %, or 5 to 10 wt %, based on the total weight of the emulsion composition.

In an exemplary embodiment, the non-amphipathic nanoparticle dispersion and the emulsion may be included in an amount of 5 wt % or more and less than 25 wt % and more than 3 wt % and less than 15 wt %, respectively, based on the total weight of the emulsion composition, in order to improve the emulsion stability of the multi-type emulsion formulation and provide the desired feeling of use and low viscosity of the multi-type emulsion formulation.

In an exemplary embodiment, the dispersed phase may have an average size of 1 to 50 μm.

In another exemplary embodiment, the dispersed phase may have an average size of 1 μm or more, 2 μm or more, 3 μm or more, 4 μm or more, 5 μm or more, 6 μm or more, 7 μm or more, 8 μm or more, 9 μm or more, or 10 μm or more, and 50 μm or less, 45 μm or less, 40 um or less, 35 μm or less, 30 μm or less, 25 μm or less, 20 μm or less, 15 μm or less, 10 μm or less, or 5 μm or less. For example, the dispersed phase may have an average size of 1 to 30 μm, 5 to 30 μm, 10 to 40 μm, or 10 to 30 μm.

In an exemplary embodiment, the size may refer to the diameter of the particle.

In an exemplary embodiment, the diameter may refer to the longest diameter.

Generally, the dispersed phase formed by emulsification with a surfactant has a small size of about 1 to 2 μm, and has a disadvantage of causing coalescence between dispersed phases. Although the Pickering emulsion composition may form a dispersed phase with a size of about 10 to 20 μm, it has a disadvantage in that it is difficult to have a stable formulation. In one aspect, the multi-type emulsion composition according to the present disclosure has an advantage in that it may have a large dispersed phase similar to the Pickering emulsion composition, while ameliorating the disadvantage of the Pickering emulsion composition having low emulsion stability in the related art, and does not cause coalescence between dispersed phases.

In the emulsion composition in the related art, the size of the dispersed phase is generally made small and uniform to prevent the coalescence of the dispersed phase in order to improve formulation stability. In contrast, the multi-type emulsion composition according to one aspect of the present disclosure has the advantage of exhibiting excellent formulation stability even when the size of the dispersed phase is large.

In an exemplary embodiment, the dispersed phase may be surrounded by non-amphipathic nanoparticles that are attached to each other at the surface of the dispersed phase.

A multi-type emulsion composition according to one aspect of the present disclosure may be one in which the sizes of the dispersed phase and non-amphipathic nanoparticles are adjusted, and due to the difference in size, the non-amphipathic nanoparticles are positioned on the surface of the dispersed phase, and an interface is formed between the dispersed phase and the continuous phase. The non-amphipathic nanoparticles having a smaller particle size compared to the dispersed phase are attracted to the surface of the dispersed phase to form an interface between the dispersed phase and the continuous phase, and the attractive forces between the non-amphipathic nanoparticles having a small particle size cause the non-amphipathic nanoparticles to be attached to each other, forming an interface surrounding the surface of the dispersed phase.

In the multi-type emulsion composition of the present disclosure according to one aspect, aqueous dispersion-type non-amphipathic nanoparticles included in the water part gather on the surface of the dispersed phase during the emulsification process, are attached to each other, and surround the dispersed phase to form an interface. Since the non-amphipathic nanoparticle dispersion is not miscible with the oil part, the non-amphipathic nanoparticles surround the dispersed phase. Therefore, unlike Pickering emulsion compositions in the related art, the multi-type emulsion composition according to the present disclosure may use the nanoparticles without surface-treating them into a Janus structure, and may implement a stable emulsification system without adding an additional surfactant other than the surfactant used in the preparation of the non-amphipathic nanoparticle dispersion and/or the emulsion.

In an exemplary embodiment, the emulsion composition may not include any additional surfactant for emulsifying the multi-type emulsion composition, except for the surfactant used in the preparation of the non-amphipathic nanoparticle dispersion and the emulsion.

In an exemplary embodiment, the emulsion composition may further include an additional surfactant for emulsifying the multi-type emulsion composition, in addition to the surfactant used in the preparation of the non-amphipathic nanoparticle dispersion and the emulsion.

The surfactant refers to a substance having two different properties that allows two or more liquids that are not miscible with each other, such as an oil phase and a water phase, to be mixed with each other.

In an exemplary embodiment, the emulsion composition may be PEG-free. The present disclosure has an advantage in that it is possible to form a low-viscosity multi-type emulsion formulation without using a PEG surfactant.

In an exemplary embodiment, the emulsion composition may have a viscosity of 4,000 to 14,000 cps at room temperature. In the related art, there was a technical limitation that it was difficult to form a multi-type emulsion formulation with a low viscosity of 10,000 cps or less, or 4,000 to 6,000 cps. Since the stability of the multi-type emulsion formulation is low, the multi-type emulsion formulation was able to be implemented only as a cream type with a high hardness in order to control the fluidity of w/o or w/s particles. In contrast, the emulsion composition according to the present disclosure has an effect of enabling a low-viscosity multi-type emulsion formulation to be formed without or with a minimum of a surfactant, an oil gelling agent and/or a thickener when forming a multi-type emulsion formulation.

In another exemplary embodiment, the emulsion composition may have a viscosity of 4,000 cps or more, 4,500 cps or more, 5,000 cps or more, 5,500 cps or more, 6,000 cps or more, 6,500 cps or more, 7,000 cps or more, 7,500 cps or more, or 8,000 cps or more, and 14,000 cps or less, 13,000 cps or less, 12,000 cps or less, 11,000 cps or less, 10,000 cps or less, 9,000 cps or less, 8,000 cps or less, 7,000 cps or less, 6,000 cps or less, 5,500 cps or less, 5,000 cps or less, or 4,500 cps or less.

In still another exemplary embodiment, when the emulsion composition is a w/o/w formulation, the viscosity may be 4,000 to 10,000 cps.

In yet another exemplary embodiment, when the emulsion composition is a w/s/w formulation, the viscosity may be 8,000 to 14,000 cps.

In an exemplary embodiment, the emulsion composition may be one in which the water part and the oil part are mixed and emulsified at room temperature.

In an exemplary embodiment, the room temperature may be 1 to 35° C., 10 to 30° C., or 15 to 25° C.

In the w/o/w multi-type emulsion formulation in the related art, when an o/w part is prepared, it is generally prepared by performing an emulsification process at a high temperature (70 to 80° C.) due to the use of an oil phase ingredient, a solid phase ingredient (for example, butter, wax, and higher alcohol), and/or an emulsifier. After preparing and mixing an oil part and a water part, a w/o part as an internal phase was added thereto, and the resulting mixture was warmed to form a w/o/w multi-type emulsion formulation. During the preparation of a multi-type emulsion formulation, it is possible to selectively compose raw materials with ingredients that need not be dissolved at high temperatures (that is, ingredients that are in a liquid state at room temperature), but in such a case, there is no way to escape from the monotonous feeling of use, so that it is not possible to provide the unique feeling of use of multi-type emulsion formulations, and the types of emulsifiers that can be used are very limited. A lamellar structure may be used to improve the stability of the multi-type structure, but the components used to make the lamellar structure also need to be dissolved under high temperature conditions. In contrast, the multi-type emulsion composition according to the present disclosure can be prepared through room temperature emulsification by mixing and emulsifying the water part and the oil part at room temperature, and heated emulsification may be selected, if necessary. Therefore, the present disclosure has an effect of significantly reducing thermal damage to an effective ingredient such as retinol, which is vulnerable to heat, as emulsification can be finally performed at room temperature during the preparation of a multi-type emulsion formulation.

In an exemplary embodiment, the emulsion composition may be a w/o/w or w/s/w formulation.

In an exemplary embodiment, the emulsion composition may be a cosmetic composition.

In an exemplary embodiment, the cosmetic composition may be a formulation such as a mist, a spray, a toner, an essence, a gel, a lotion, a cream, a pack, foam cleanser, a makeup base, and a foundation.

In an exemplary embodiment, the emulsion composition may be a composition for external use on the skin. The composition for external use on the skin means something that is applied externally to the skin, and may include, for example, pharmaceuticals in various formulations. In an exemplary embodiment, the composition for external use on the skin may be a formulation such as a mist, a spray, a suspension, an emulsion, a gel, a lotion, and an ointment.

In another aspect, the present disclosure provides a method for preparing the multi-type emulsion composition, the method including: preparing a non-amphipathic nanoparticle dispersion; preparing an emulsion including an oil phase or silicone phase as an external phase; preparing a water part including the non-amphipathic nanoparticle dispersion; preparing an oil part including the emulsion; and mixing the water part and the oil part.

In the preparation method, the order in which the non-amphipathic nanoparticle dispersion and the emulsion are prepared and/or the order in which the water part and the oil part are prepared can be arbitrarily selected by those skilled in the art.

In an exemplary embodiment, in the preparation method, a surfactant may be further added in the process of mixing the water part and the oil part.

In an exemplary embodiment, the preparation method may mix and emulsify the water part and the oil part at room temperature.

Hereinafter, the present disclosure will be described in more detail through examples. These examples are only for exemplifying the present disclosure, and it will be obvious to those of ordinary skill in the art that the scope of the present disclosure should not be construed as being limited by these examples.

### Preparation Example 1. Preparation of Non-Amphipathic Nanoparticle Dispersion

According to the composition in the following Table 1, an aqueous dispersion containing nanoemulsion particles as non-amphipathic nanoparticles was prepared. An oil phase was heated to 70° C. and melted, then dispersed using a homogenizer to prepare a lipophilic mixture. A water phase was heated and dissolved at 70° C. in a separate container, and then the lipophilic mixture prepared above was slowly added to the water phase, and a homogenizer was used at 70° C. to form aqueous dispersion-type nanoparticles. The size of the nanoparticles was adjusted using an ultrasonic and high pressure homogenizer as the homogenizer.

TABLE 1

| | Preparation Example 1-1 |
|---|---|
| | (wt %) |
| WATER | To 100 |
| PROPANEDIOL | 7.5 |
| 1,2-HEXANDIOL | 1 |
| ETHYLHEXYLGLYCERIN | 0.05 |
| GLYCERIN | 10 |
| STEARIC ACID | 0.2 |
| PALMITIC ACID | 0.2 |
| TOCOPHEROL | 0.5 |
| PHYTOSTERYL/BEHENYL/ | 1 |
| OCTYLDODECYL LAUROYL | |
| GLUTAMATE | |
| SHEA BUTTER | 5 |
| HYDROGENATED LECITHIN | 2 |
| MEADOWFOAM SEED OIL | 3 |

According to the composition of the following Table 2, an aqueous dispersion containing solid lipid nanoparticles as non-amphipathic nanoparticles was prepared. An oil phase was heated to 70° C. and melted, then dispersed using a homogenizer to prepare a lipophilic mixture. A water phase was heated and dissolved at 70° C. in a separate container, and then the lipophilic mixture prepared above was slowly added to the water phase, and a homogenizer was used at 70° C. to form aqueous dispersion-type nanoparticles. Upon cooling, recrystallization of the lipids occurs and nanoparticles are formed. The size of the nanoparticles was adjusted using an ultrasonic and high pressure homogenizer as the homogenizer.

TABLE 2

| | Preparation Example 1-2 |
|---|---|
| | (wt %) |
| WATER | To 100 |
| BUTYLENE GLYCOL | 20 |
| 1,2-HEXANDIOL | 1 |
| SQUALANE | 8 |
| ETHYLHEXYLGLYCERIN | 0.05 |
| CERAMIDE NP | 0.1 |
| STEARIC ACID | 0.05 |
| PALMITIC ACID | 0.05 |
| TOCOPHEROL | 0.1 |
| CHOLESTEROL | 0.1 |
| POLYGLYCERYL-10 STEARATE | 2 |
| SYNTHETIC WAX | 10 |
| HYDROGENATED LECITHIN | 2 |
| PANTHENOL | 1.5 |

According to the composition in the following Table 3, an aqueous dispersion containing liposomes as non-amphipathic nanoparticles was prepared. A mixed solution was prepared by dissolving 100% hydrogenated oleoyl-palmitoyl/oleoyl-stearyl phosphatidylcholine mixture (Lipoid S100-3) and cholesterol in ethanol with heating. After the mixed solution was poured into water at 60° C., and then mixed and stirred at 5,000 rpm for 5 minutes using a homogenizer, aqueous dispersion-type nanoparticles were prepared using a high pressure homogenizer (1,000 bar, 3 cycles). A rotary evaporator was used to remove the remaining ethanol solution and prepare liposomes composed of lipid-cholesterol.

TABLE 3

| | Preparation |
|---|---|
| | Example 1-3 (g) |
| CHOLESTEROL | 0.67 |
| MIXTURE of OLEOYL-PALMITOYL/OLEOYL- | 5.33 |
| STEARYL PHOSPHATIDYLCHOLINE | |
| (Lipoid S100-3) | |
| ETHANOL | 30 |
| WATER | 300 |
| Total weight after distillation | 100 |

According to the composition in the following Table 4, an aqueous dispersion containing polymersomes as non-amphipathic nanoparticles was prepared. Poly(methacrylic acid-co-stearyl methacrylate) copolymer, 100% hydrogenated oleoyl-palmitoyl/oleoyl-stearyl phosphatidylcholine mixture (Lipoid S100-3), and cholesterol were dissolved in ethanol at 60° C. with heating to prepare a mixed solution. After the mixed solution was poured into water at 60° C., and then mixed and stirred at 5,000 rpm for 5 minutes using a homogenizer to prepare a primary dispersed polymer-liposome composite, aqueous dispersion-type nanoparticles were prepared using a high pressure homogenizer (1,000 bar, 3 cycles). A rotary evaporator was used to remove the remaining ethanol solution and prepare a polymer-liposome nanocomposite using an acidity-sensitive polymer.

TABLE 4

| | Preparation |
|---|---|
| | Example 1-4 (g) |
| POLY(METHACRYLIC ACID-co-STEARYL | 1.8 |
| METHACRYLATE) COPOLYMER | |
| MIXTURE of OLEOYL-PALMITOYL/OLEOYL- | 3.15 |
| STEARYL PHOSPHATIDYLCHOLINE | |
| (Lipoid S100-3) | |
| CHOLESTEROL | 1.05 |
| ETHANOL | 30 |
| WATER | 300 |
| Total weight after distillation | 100 |

### Preparation Example 2. Preparation of Emulsion

A w/o emulsion was prepared according to the composition in the following Table 5. An oil phase was prepared and uniformly dispersed at 70° C. using an Agi mixer. A water phase was prepared, heated to 70° C., and stirred. The water phase was added to the oil phase to perform emulsification at 4,000 rpm for 5 minutes using a homogenizer, and then the resulting emulsion was cooled.

TABLE 5

| | | Preparation Example 2-1 |
|---|---|---|
| | | (wt %) |
| Oil | SQUALANE | 25 |
| phase | PHYTOSTERYL/BEHENYL/OCTYLDODECYL | 1 |
| | LAUROYL GLUTAMATE*TOCOPHEROL | |
| | CETYL PEG/PPG-10/1 | 2 |
| | DIMETHICONE*PENTAERYTHRITYL | |
| | TETRA-DI-T-BUTYL | |
| | HYDROXYHYDROCINNAMATE | |
| | LAURYL PEG/PPG-18/18 | 0.3 |
| | METHICONE*ISOSTEARYL ALCOHOL | |
| | PEG-30 DIPOLYHYDROXYSTEARATE | 2 |
| Water | WATER | To 100 |
| phase | ACETYL GLUCOSAMINE | 1 |

A w/o emulsion was prepared according to the composition in the following Table 6. An oil phase was prepared and uniformly dispersed at 70° C. using an Agi mixer. A water phase was prepared, heated to 70° C., and stirred. The water phase was added to the oil phase to perform emulsification at 4,000 rpm for 5 minutes using a homogenizer, and then the resulting emulsion was cooled.

TABLE 6

| | | Preparation Example 2-2 |
|---|---|---|
| | | (wt %) |
| Oil | POLYGLYCERYL-4 OLEATE*POLYGLYCERYL- | 4 |
| phase | 3 POLYRICINOLEATE | |
| | POLYGLYCERYL-3 POLYRICINOLEATE | 1 |
| | ETHYL OLIVATE | 10 |
| | DICAPRYLYL ETHER*TOCOPHEROL | 10 |
| Water | WATER | To 100 |
| phase | CAPRYLYL GLYCOL | 0.2 |
| | 1,2-HEXANDIOL | 1.5 |
| | MAGNESIUM SULFATE | 2 |
| | GLYCERIN | 2 |
| | BUTYLENE GLYCOL | 2 |

A w/s emulsion was prepared according to the composition in the following Table 7. A silicone phase was prepared and uniformly dispersed at room temperature using an Agi mixer. A water phase was prepared and uniformly dissolved and dispersed using an Agi mixer. The water phase was added to the silicone phase to perform emulsification at 2,000 rpm for 5 5 minutes using a homogenizer.

TABLE 7

| | | Preparation Example 2-3 |
|---|---|---|
| | | (wt %) |
| Silicone | DIMETHICONE*PEG-15/LAURYL | 1.5 |
| phase | DIMETHICONE | |
| | CROSSPOLYMER*TOCOPHEROL | |
| | DIMETHICONE*DIMETHICONE/VINYL | 1.5 |
| | DIMETHICONE CROSSPOLYMER | |
| | DIMETHICONE*POLYSILICONE-11 | 1 |
| | LAURYL PEG-9 | 1 |
| | POLYDIMETHYLSILOXYETHYL | |
| | DIMETHICONE*TOCOPHEROL | |
| | PEG-10 DIMETHICONE | 0.5 |
| | DIMETHICONE | 8 |
| | ISODODECANE | 2 |
| | POLYMETHYLSILSESQUIOXANE | 0.1 |
| Water | WATER | To 100 |
| phase | BETAINE | 1 |
| | MAGNESIUM SULFATE | 0.5 |
| | SODIUM CHLORIDE | 0.5 |
| | GLYCERIN | 7 |
| | DIPROPYLENE GLYCOL | 5 |
| | 1,2-HEXANDIOL | 1.5 |
| | ETHYLHEXYLGLYCERIN*TOCOPHEROL | 0.1 |
| | HYDROXYPROPYL STARCH PHOSPHATE | 0.1 |

### Example 1. Preparation of W/O/W Multi-Type Emulsion Composition

W/O/W multi-type emulsion compositions emulsified with the compositions (wt %) in the following Tables 8 to 10 at room temperature were prepared. A water part, in which a non-amphipathic nanoparticle aqueous dispersion was excluded, was prepared, and the water part was stirred at 8,000 rpm for 3 minutes with a homogenizer and sufficiently dispersed at room temperature. A non-amphipathic nanoparticle aqueous dispersion was added to the dispersed water part, and the mixture was sufficiently stirred using an Agi mixer to prepare a water part containing the non-amphipathic nanoparticle aqueous dispersion. In addition, an oil part, in which an emulsion was excluded, was uniformly mixed at room temperature, and then a prepared emulsion was added to prepare an oil part containing the emulsion. Thereafter, the oil part was added to the water part to perform emulsification at room temperature for 3 to 5 minutes at 2,000 rpm using a homogenizer, thereby preparing a multi-type emulsion composition.

As a result of measuring the viscosity of the emulsion compositions of Examples 1-1 to 1-15 at room temperature, it was confirmed that the viscosity was in a range of 6500±500 cps (measuring apparatus: Viscometer, LVDV-II+Pro, Brookfield, USA).

In addition, each emulsion composition was stored at room temperature, 37° C., 45° C., 60° C., under cycling conditions (a method in which the temperature was changed from 45° C. to −15° C. or from −15° C. to 45° C. in a 12-hour cycle), under refrigerated (5° C.) and frozen (−15° C.) conditions, and emulsion stability was evaluated with the naked eye after 4 weeks. As a result, it was found that the emulsion compositions of Examples 1-1 to 1-15 had formulation stability without separation of the oil part and the water part. The evaluation criteria are as follows: ⊚; the case where the interface between the water part and the oil part is well maintained under all temperature conditions and there is no oil separation phenomenon, [symbol] ; the case where the interface between the water part and the oil part is partially unstable under temperature conditions of 1 or more, X; the case where the interface between the water part and the oil part cracks and oil separation phenomenon is observed under temperature conditions of 1 or more.

TABLE 8

| | | Example | Example | Example | Example | Example | Example |
|---|---|---|---|---|---|---|---|
| | | 1-1 | 1-2 | 1-3 | 1-4 | 1-5 | 1-6 |
| Oil | BUTYLENE GLYCOL | 2 | 2 | 2 | 2 | 2 | 2 |
| part | DICAPRYLATE/DICAPRATE | | | | | | |
| | SOYBEAN OIL*RETINOL | 1 | 1 | 1 | 1 | 1 | 1 |
| | CAPRYLIC/CAPRIC/ | 3 | 3 | 3 | 3 | 3 | 3 |
| | MYRISTIC/STEARIC | | | | | | |
| | TRIGLYCERIDE | | | | | | |
| | TRIDECYL | 2 | 2 | 2 | 2 | 2 | 2 |
| | TRIMELLITATE | | | | | | |
| | C9-12 ALKANE | 2 | 2 | 2 | 2 | 2 | 2 |
| | Emulsion of Preparation | 10 | 10 | 10 | | | |
| | Example 2-1 | | | | | | |
| | Emulsion of Preparation | | | | 10 | 10 | 10 |
| | Example 2-2 | | | | | | |
| Water | WATER | To | To | To | To | To | To |
| part | | 100 | 100 | 100 | 100 | 100 | 100 |
| | ADENOSINE | 0.04 | 0.04 | 0.04 | 0.04 | 0.04 | 0.04 |
| | SORBITOL*WATER/ | 5 | 5 | 5 | 5 | 5 | 5 |
| | AQUA/EAU*MALTITOL | | | | | | |
| | 2,3-BUTANEDIOL | 3 | 3 | 3 | 3 | 3 | 3 |
| | PENTYLENE GLYCOL | 1 | 1 | 1 | 1 | 1 | 1 |
| | GLYCERYL CAPRYLATE | 0.1 | 0.1 | 0.1 | 0.1 | 0.1 | 0.1 |
| | ETHYLHEXYLGLYCERIN*TO- | 0.05 | 0.05 | 0.05 | 0.05 | 0.05 | 0.05 |
| | COPHEROL | | | | | | |
| | WATER/AQUA/ | 3 | 3 | 3 | 3 | 3 | 3 |
| | EAU*CELLULOSE*PENTYLENE | | | | | | |
| | GLYCOL*GLYCERYL | | | | | | |
| | CAPRYLATE | | | | | | |
| | SPHINGOMONAS | 0.05 | 0.05 | 0.05 | 0.05 | 0.05 | 0.05 |
| | FERMENT EXTRACT | | | | | | |
| | POLYACRYLATE | 0.1 | 0.1 | 0.1 | 0.1 | 0.1 | 0.1 |
| | CROSSPOLYMER-6 | | | | | | |
| | FRAGRANCE | 0.1 | 0.1 | 0.1 | 0.1 | 0.1 | 0.1 |
| | AMMONIUM | 0.1 | 0.1 | 0.1 | 0.1 | 0.1 | 0.1 |
| | POLYACRYLOYLDIMETHYL | | | | | | |
| | TAURATE | | | | | | |
| | XANTHAN GUM | 0.03 | 0.03 | 0.03 | 0.03 | 0.03 | 0.03 |
| | Non-amphipathic | 15 | | 7.5 | 15 | | 7.5 |
| | nanoparticle aqueous | | | | | | |
| | dispersion of Preparation | | | | | | |
| | Example 1-1 | | | | | | |
| | Non-amphipathic | | 15 | 7.5 | | 15 | 7.5 |
| | nanoparticle aqueous | | | | | | |
| | dispersion of Preparation | | | | | | |
| | Example 1-2 | | | | | | |
| | Stability (based on 4 weeks) | ⊚ | ⊚ | ⊚ | ⊚ | ⊚ | ⊚ |

TABLE 9

| | | Example | Example | Example | Example | Example |
|---|---|---|---|---|---|---|
| | | 1-7 | 1-8 | 1-9 | 1-10 | 1-11 |
| Oil | BUTYLENE GLYCOL | 2 | 2 | 2 | 2 | 2 |
| part | DICAPRYLATE/DICAPRATE | | | | | |
| | SOYBEAN OIL*RETINOL | 1 | 1 | 1 | 1 | 1 |
| | CAPRYLIC/CAPRIC/ | 3 | 3 | 3 | 3 | 3 |
| | MYRISTIC/STEARIC | | | | | |
| | TRIGLYCERIDE | | | | | |
| | TRIDECYL | 2 | 2 | 2 | 2 | 2 |
| | TRIMELLITATE | | | | | |
| | C9-12 ALKANE | 2 | 2 | 2 | 2 | 2 |
| | Emulsion of Preparation | 13 | 13 | 13 | 5 | |
| | Example 2-1 | | | | | |
| | Emulsion of Preparation | | | | | 5 |
| | Example 2-2 | | | | | |
| Water | WATER | To | To | To | To | To |
| part | | 100 | 100 | 100 | 100 | 100 |
| | ADENOSINE | 0.04 | 0.04 | 0.04 | 0.04 | 0.04 |
| | SORBITOL*WATER/ | 5 | 5 | 5 | 5 | 5 |
| | AQUA/EAU*MALTITOL | | | | | |
| | 2,3-BUTANEDIOL | 3 | 3 | 3 | 3 | 3 |
| | PENTYLENE GLYCOL | 1 | 1 | 1 | 1 | 1 |
| | GLYCERYL CAPRYLATE | 0.1 | 0.1 | 0.1 | 0.1 | 0.1 |
| | ETHYLHEXYLGLYCERIN*TO- | 0.05 | 0.05 | 0.05 | 0.05 | 0.05 |
| | COPHEROL | | | | | |
| | WATER/AQUA/ | 3 | 3 | 3 | 3 | 3 |
| | EAU*CELLULOSE*PENTYLENE | | | | | |
| | GLYCOL*GLYCERYL | | | | | |
| | CAPRYLATE | | | | | |
| | SPHINGOMONAS | 0.05 | 0.05 | 0.05 | 0.05 | 0.05 |
| | FERMENT EXTRACT | | | | | |
| | POLYACRYLATE | 0.1 | 0.1 | 0.1 | 0.1 | 0.1 |
| | CROSSPOLYMER-6 | | | | | |
| | FRAGRANCE | 0.1 | 0.1 | 0.1 | 0.1 | 0.1 |
| | AMMONIUM | 0.1 | 0.1 | 0.1 | 0.1 | 0.1 |
| | POLYACRYLOYLDIMETHYL | | | | | |
| | TAURATE | | | | | |
| | XANTHAN GUM | 0.03 | 0.03 | 0.03 | 0.03 | 0.03 |
| | Non-amphipathic | 20 | | | 15 | 15 |
| | nanoparticle aqueous | | | | | |
| | dispersion of Preparation | | | | | |
| | Example 1-1 | | | | | |
| | Non-amphipathic | | | | | |
| | nanoparticle aqueous | | | | | |
| | dispersion of Preparation | | | | | |
| | Example 1-2 | | | | | |
| | Non-amphipathic | | 20 | | | |
| | nanoparticle aqueous | | | | | |
| | dispersion of Preparation | | | | | |
| | Example 1-3 | | | | | |
| | Non-amphipathic | | | 20 | | |
| | nanoparticle aqueous | | | | | |
| | dispersion of Preparation | | | | | |
| | Example 1-4 | | | | | |
| | Stability (based on 4 weeks) | ⊚ | ⊚ | ⊚ | ⊚ | ⊚ |

TABLE 10

| | | Example | Example | Example | Example |
|---|---|---|---|---|---|
| | | 1-12 | 1-13 | 1-14 | 1-15 |
| Oil | BUTYLENE GLYCOL | 2 | 2 | 2 | 2 |
| part | DICAPRYLATE/DICAPRATE | | | | |
| | SOYBEAN OIL*RETINOL | 1 | 1 | 1 | 1 |
| | CAPRYLIC/CAPRIC/ | 3 | 3 | 3 | 3 |
| | MYRISTIC/STEARIC | | | | |
| | TRIGLYCERIDE | | | | |
| | TRIDECYL | 2 | 2 | 2 | 2 |
| | TRIMELLITATE | | | | |
| | C9-12 ALKANE | 2 | 2 | 2 | 2 |
| | Emulsion of | 5 | 5 | | |
| | Preparation | | | | |
| | Example 2-1 | | | | |
| | Emulsion of | | | 5 | 5 |
| | Preparation | | | | |
| | Example 2-2 | | | | |
| Water | WATER | To 100 | To 100 | To 100 | To 100 |
| part | ADENOSINE | 0.04 | 0.04 | 0.04 | 0.04 |
| | SORBITOL*WATER/ | 5 | 5 | 5 | 5 |
| | AQUA/EAU*MALTITOL | | | | |
| | 2,3-BUTANEDIOL | 3 | 3 | 3 | 3 |
| | PENTYLENE GLYCOL | 1 | 1 | 1 | 1 |
| | GLYCERYL CAPRYLATE | 0.1 | 0.1 | 0.1 | 0.1 |
| | ETHYLHEXYL- | 0.05 | 0.05 | 0.05 | 0.05 |
| | GLYCERIN*TOCOPHEROL | | | | |
| | WATER/AQUA/ | 3 | 3 | 3 | 3 |
| | EAU*CELLULOSE*PENTYLENE | | | | |
| | GLYCOL*GLYCERYL | | | | |
| | CAPRYLATE | | | | |
| | SPHINGOMONAS | 0.05 | 0.05 | 0.05 | 0.05 |
| | FERMENT EXTRACT | | | | |
| | POLYACRYLATE | 0.1 | 0.1 | 0.1 | 0.1 |
| | CROSSPOLYMER-6 | | | | |
| | FRAGRANCE | 0.1 | 0.1 | 0.1 | 0.1 |
| | AMMONIUM | 0.1 | 0.1 | 0.1 | 0.1 |
| | POLYACRYLOYLDIMETHYL | | | | |
| | TAURATE | | | | |
| | XANTHAN GUM | 0.03 | 0.03 | 0.03 | 0.03 |
| | Non-amphipathic | 10 | | 10 | |
| | nanoparticle aqueous | | | | |
| | dispersion of | | | | |
| | Preparation | | | | |
| | Example 1-1 | | | | |
| | Non-amphipathic | | 10 | | 10 |
| | nanoparticle aqueous | | | | |
| | dispersion of | | | | |
| | Preparation | | | | |
| | Example 1-2 | | | | |
| | Stability | ⊚ | ⊚ | ⊚ | ⊚ |
| | (based on 4 weeks) | | | | |

As confirmed above, the present disclosure formed a multi-type emulsion composition by mixing and emulsifying an oil part containing an emulsion and a water part containing a non-amphipathic nanoparticle dispersion. It was confirmed that the emulsion compositions of Examples 1-1 to 1-15, in which the emulsion content was less than 15 wt % based on the total weight of the emulsion composition and the non-amphipathic nanoparticle dispersion was contained at a weight ratio of more than 1 with respect to the emulsion content, significantly increased the stability of the thermodynamically unstable multi-type emulsion formulation. Further, it was confirmed that emulsions with and without PEG formed a multi-type emulsion composition with excellent formulation stability.

Meanwhile, when ingredients vulnerable to heat (for example, retinol) are added to multi-type emulsion formulations in the related art, there is a technical limitation in that there is no method of preventing exposure to heat. This is because a warming process is used when an external phase o/w part is produced in order to produce the final w/o/w multi-type emulsion formulation. In contrast, it was confirmed that in the present disclosure, emulsification can be performed at room temperature during the preparation of a multi-type emulsion formulation, thereby significantly reducing thermal damage to retinol ingredients vulnerable to heat.

### Example 2. Preparation of W/S/W Multi-Type Emulsion Composition

According to the method described in Example 1 above, w/s/w multi-type emulsion compositions were prepared with the compositions (wt %) shown in the following Table 11, and the viscosity and emulsion stability were evaluated.

As a result of measuring the viscosity of the emulsion compositions of Examples 2-1 and 2-2, it was confirmed that the viscosity was in a range of 11,000±2,000 cps.

Furthermore, it was confirmed that a w/s/w multi-type emulsion composition with excellent formulation stability can be formed by mixing and emulsifying an oil part containing a w/s emulsion and a water part containing a non-amphipathic nanoparticle dispersion.

TABLE 11

| | | Example | Example |
|---|---|---|---|
| | | 2-1 | 2-2 |
| Oil | SQUALANE | 3 | 3 |
| part | DIMETHICONE | 5 | 5 |
| | CYCLOPENTASILOXANE*DI- | 1 | 1 |
| | METHICONE*DIMETHICONE/ | | |
| | PEG-10/15 CROSSPOLYMER | | |
| | Emulsion of Preparation | 7 | 7 |
| | Example 2-3 | | |
| Water | WATER | To 100 | To 100 |
| part | ADENOSINE | 0.04 | 0.04 |
| | SORBITOL*WATER/AQUA/ | 5 | 5 |
| | EAU*MALTITOL | | |
| | 2,3-BUTANEDIOL | 3 | 3 |
| | PENTYLENE GLYCOL | 1 | 1 |
| | GLYCERYL CAPRYLATE | 0.1 | 0.1 |
| | ETHYLHEXYLGLYCERIN*TOCOPHEROL | 0.05 | 0.05 |
| | WATER/AQUA/ | 3 | 3 |
| | EAU*CELLULOSE*PENTYLENE | | |
| | GLYCOL*GLYCERYL CAPRYLATE | | |
| | SPHINGOMONAS FERMENT EXTRACT | 0.05 | 0.05 |
| | POLYACRYLATE CROSSPOLYMER-6 | 0.15 | 0.15 |
| | FRAGRANCE | 0.1 | 0.1 |
| | AMMONIUM | 0.2 | 0.2 |
| | POLYACRYLOYLDIMETHYL | | |
| | TAURATE | | |
| | XANTHAN GUM | 0.1 | 0.1 |
| | Non-amphipathic | 15 | |
| | nanoparticle aqueous | | |
| | dispersion of | | |
| | Preparation | | |
| | Example 1-1 | | |
| | Non-amphipathic | | 15 |
| | nanoparticle aqueous | | |
| | dispersion of | | |
| | Preparation | | |
| | Example 1-2 | | |
| | Stability | ⊚ | ⊚ |
| | (based on 4 weeks) | | |

### Comparative Example 1. Preparation of W/O/W Multi-Type Emulsion Composition

According to the method described in Example 1 above, w/o/w multi-type emulsion compositions were prepared with the compositions (wt %) shown in the following Tables 12 and 13, and the viscosity and emulsion stability were evaluated.

As a result of measuring the viscosity of the emulsion compositions of Comparative Examples 1-1 to 1-9, it was confirmed that the viscosity of the emulsion compositions of Comparative Examples 1-1 to 1-8 was in a range of 6500±500 cps, and the viscosity of the emulsion composition of Comparative Example 1-9 exceeded 14,000 cps.

It was confirmed that the emulsion compositions of Comparative Examples 1-1 to 1-4, in which the emulsion content was less than 15 wt % based on the total weight of the emulsion composition and the non-amphipathic nanoparticle dispersion was contained at a weight ratio of more than 1 with respect to the emulsion content, exhibited excellent formulation stability. However, since the emulsion compositions of Comparative Examples 1-1 to 1-4 contained 3 wt % or less of the emulsion, it was found that they could not provide the feeling of use characteristic of the multi-type. The multi-type emulsion formulation has the characteristics that while emulsion particles burst, the water on the outermost phase, the oil particles in the middle phase, and the water on the innermost phase are sequentially felt, providing a variety of feelings of use. Therefore, it was confirmed that in forming the multi-type emulsion composition according to the present disclosure, it is preferable to use the emulsion in an amount exceeding 3 wt %.

It was confirmed that the emulsion compositions of Comparative Examples 1-5 to 1-8 contained a high content of emulsion of 15 wt %, and thus exhibited a heavy and thick feeling of use. In addition, since the emulsion compositions of Comparative Examples 1-5 to 1-8 contained the emulsion and the non-amphipathic nanoparticle dispersion at a weight ratio of 1:1, the content of the non-amphipathic nanoparticle dispersion was low, so that it was found that the emulsion stability of the multi-type emulsion formulation deteriorated.

It was found that the emulsion composition of Comparative Examples 1-9, in which the emulsion content was less than 15 wt % based on the total weight of the emulsion composition and the non-amphipathic nanoparticle dispersion was contained at a weight ratio of more than 1 with respect to the emulsion content, exhibited excellent formulation stability. However, since the emulsion composition of Comparative Example 1-9 containing 25 wt % of the non-amphipathic nanoparticle dispersion had a viscosity exceeding 14,000 cps, it was unable to form a low-viscosity w/o/w formulation. Therefore, it was confirmed that in forming the low-viscosity multi-type emulsion composition according to the present disclosure, it is desirable to use the non-amphipathic nanoparticle dispersion in an amount of less than 25 wt %.

TABLE 12

| | | Comparative | Comparative | Comparative | Comparative |
|---|---|---|---|---|---|
| | | Example | Example | Example | Example |
| | | 1-1 | 1-2 | 1-3 | 1-4 |
| Oil | BUTYLENE GLYCOL | 2 | 2 | 2 | 2 |
| part | DICAPRYLATE/DICAPRATE | | | | |
| | SOYBEAN OIL*RETINOL | 1 | 1 | 1 | 1 |
| | CAPRYLIC/CAPRIC/ | 3 | 3 | 3 | 3 |
| | MYRISTIC/STEARIC | | | | |
| | TRIGLYCERIDE | | | | |
| | TRIDECYL | 2 | 2 | 2 | 2 |
| | TRIMELLITATE | | | | |
| | C9-12 ALKANE | 2 | 2 | 2 | 2 |
| | Emulsion of Preparation | 2 | 3 | | |
| | Example 2-1 | | | | |
| | Emulsion of Preparation | | | 2 | 3 |
| | Example 2-2 | | | | |
| Water | WATER | To 100 | To 100 | To 100 | To 100 |
| part | ADENOSINE | 0.04 | 0.04 | 0.04 | 0.04 |
| | SORBITOL*WATER/ | 5 | 5 | 5 | 5 |
| | AQUA/EAU*MALTITOL | | | | |
| | 2,3-BUTANEDIOL | 3 | 3 | 3 | 3 |
| | PENTYLENE GLYCOL | 1 | 1 | 1 | 1 |
| | GLYCERYL CAPRYLATE | 0.1 | 0.1 | 0.1 | 0.1 |
| | ETHYLHEXYL- | 0.05 | 0.05 | 0.05 | 0.05 |
| | GLYCERIN*TOCOPHEROL | | | | |
| | WATER/AQUA/ | 3 | 3 | 3 | 3 |
| | EAU*CELLULOSE*PENTYLENE | | | | |
| | GLYCOL*GLYCERYL | | | | |
| | CAPRYLATE | | | | |
| | SPHINGOMONAS | 0.05 | 0.05 | 0.05 | 0.05 |
| | FERMENT EXTRACT | | | | |
| | POLYACRYLATE | 0.1 | 0.1 | 0.1 | 0.1 |
| | CROSSPOLYMER-6 | | | | |
| | FRAGRANCE | 0.1 | 0.1 | 0.1 | 0.1 |
| | AMMONIUM | 0.1 | 0.1 | 0.1 | 0.1 |
| | POLYACRYLOYLDIMETHYL | | | | |
| | TAURATE | | | | |
| | XANTHAN GUM | 0.03 | 0.03 | 0.03 | 0.03 |
| | Non-amphipathic | 15 | | 15 | |
| | nanoparticle aqueous | | | | |
| | dispersion of | | | | |
| | Preparation | | | | |
| | Example 1-1 | | | | |
| | Non-amphipathic | | 15 | | 15 |
| | nanoparticle aqueous | | | | |
| | dispersion of | | | | |
| | Preparation | | | | |
| | Example 1-2 | | | | |
| | Stability | ⊚ | ⊚ | ⊚ | ⊚ |
| | (based on 4 weeks) | | | | |

TABLE 13

| | | Comparative | Comparative | Comparative | Comparative | Comparative |
|---|---|---|---|---|---|---|
| | | Example | Example | Example | Example | Example |
| | | 1-5 | 1-6 | 1-7 | 1-8 | 1-9 |
| Oil | BUTYLENE GLYCOL | 2 | 2 | 2 | 2 | 2 |
| part | DICAPRYLATE/DICAPRATE | | | | | |
| | SOYBEAN OIL*RETINOL | 1 | 1 | 1 | 1 | 1 |
| | CAPRYLIC/CAPRIC/ | 3 | 3 | 3 | 3 | 3 |
| | MYRISTIC/STEARIC | | | | | |
| | TRIGLYCERIDE | | | | | |
| | TRIDECYL | 2 | 2 | 2 | 2 | 2 |
| | TRIMELLITATE | | | | | |
| | C9-12 ALKANE | 2 | 2 | 2 | 2 | 2 |
| | Emulsion of Preparation | 15 | | 15 | | |
| | Example 2-1 | | | | | |
| | Emulsion of Preparation | | 15 | | 15 | 5 |
| | Example 2-2 | | | | | |
| Water | WATER | To | To | To | To | To |
| part | | 100 | 100 | 100 | 100 | 100 |
| | ADENOSINE | 0.04 | 0.04 | 0.04 | 0.04 | 0.04 |
| | SORBITOL*WATER/ | 5 | 5 | 5 | 5 | 5 |
| | AQUA/EAU*MALTITOL | | | | | |
| | 2,3-BUTANEDIOL | 3 | 3 | 3 | 3 | 3 |
| | PENTYLENE GLYCOL | 1 | 1 | 1 | 1 | 1 |
| | GLYCERYL CAPRYLATE | 0.1 | 0.1 | 0.1 | 0.1 | 0.1 |
| | ETHYLHEXYLGLYCERIN*TO- | 0.05 | 0.05 | 0.05 | 0.05 | 0.05 |
| | COPHEROL | | | | | |
| | WATER/AQUA/ | 3 | 3 | 3 | 3 | 3 |
| | EAU*CELLULOSE*PENTYLENE | | | | | |
| | GLYCOL*GLYCERYL | | | | | |
| | CAPRYLATE | | | | | |
| | SPHINGOMONAS | 0.05 | 0.05 | 0.05 | 0.05 | 0.05 |
| | FERMENT EXTRACT | | | | | |
| | POLYACRYLATE | 0.1 | 0.1 | 0.1 | 0.1 | 0.1 |
| | CROSSPOLYMER-6 | | | | | |
| | FRAGRANCE | 0.1 | 0.1 | 0.1 | 0.1 | 0.1 |
| | AMMONIUM | 0.1 | 0.1 | 0.1 | 0.1 | 0.1 |
| | POLYACRYLOYLDIMETHYL | | | | | |
| | TAURATE | | | | | |
| | XANTHAN GUM | 0.03 | 0.03 | 0.03 | 0.03 | 0.03 |
| | Non-amphipathic | 15 | 15 | | | |
| | nanoparticle aqueous | | | | | |
| | dispersion of Preparation | | | | | |
| | Example 1-1 | | | | | |
| | Non-amphipathic | | | 15 | 15 | 25 |
| | nanoparticle aqueous | | | | | |
| | dispersion of Preparation | | | | | |
| | Example 1-2 | | | | | |
| | Stability (based on 4 weeks) | Δ | Δ | Δ | Δ | ⊚ |

Although the specific part of the present disclosure has been described in detail, it will be apparent to those of ordinary skill in the art that such a specific description is just a preferred embodiment and the scope of the present disclosure is not limited thereby. Therefore, the substantial scope of the present disclosure will be defined by the appended claims and equivalents thereof.

What is claimed is:

1. A multi-type emulsion composition, wherein the emulsion composition is a composition in which a water part and an oil part are mixed and emulsified,
the water part comprises a non-amphipathic nanoparticle dispersion,
the oil part comprises an emulsion and the emulsion comprises an oil phase or silicone phase as an external phase,
the emulsion composition comprises the water part as a continuous phase and the oil part as a dispersed phase, and
the dispersed phase is surrounded by the non-amphipathic nanoparticles.

2. The multi-type emulsion composition of claim 1, wherein the non-amphipathic nanoparticles have an average size of 10 nm or more and less than 1 μm.

3. The multi-type emulsion composition of claim 1, wherein the non-amphipathic nanoparticle is selected from the group consisting of a nanoemulsion particle, a solid lipid nanoparticle (SLN), a liposome, and a polymersome.

4. The multi-type emulsion composition of claim 1, wherein the non-amphipathic nanoparticle dispersion is immiscible with the oil part and/or immiscible with the oil phase or silicone phase.

5. The multi-type emulsion composition of claim 1, wherein the emulsion comprises a water phase as an internal phase.

6. The multi-type emulsion composition of claim 1, wherein the non-amphipathic nanoparticle dispersion and the emulsion are comprised at a weight ratio of more than 1:1.

7. The multi-type emulsion composition of claim 1, wherein the non-amphipathic nanoparticle dispersion is comprised in an amount of less than 25 wt % based on a total weight of the emulsion composition.

8. The multi-type emulsion composition of claim 1, wherein the emulsion is comprised in an amount of less than 15 wt % based on a total weight of the emulsion composition.

9. The multi-type emulsion composition of claim 1, wherein the dispersed phase has an average size of 1 to 50 μm.

10. The multi-type emulsion composition of claim 1, further comprising a surfactant.

11. The multi-type emulsion composition of claim 1, wherein the emulsion composition is PEG-free.

12. The multi-type emulsion composition of claim 1, wherein the emulsion composition has a viscosity of 4,000 to 14,000 cps.

13. The multi-type emulsion composition of claim 1, wherein the emulsion composition is a composition in which the water part and the oil part are mixed and emulsified at room temperature.

14. The multi-type emulsion composition of claim 1, wherein the emulsion composition is a w/o/w or w/s/w formulation.

15. A method for preparing the multi-type emulsion composition of claim 1, the method comprising: preparing a non-amphipathic nanoparticle dispersion;
preparing an emulsion comprising an oil phase or silicone phase as an external phase;
preparing a water part comprising the non-amphipathic nanoparticle dispersion;
preparing an oil part comprising the emulsion; and
mixing the water part and the oil part.
